(19) United States
(12) Patent Application Publication
(10) Pub. No.: US 2013/0115178 A1
(43) Pub. Date: May 9, 2013

(54) TANNING ENHANCERS AND SELF-TANNING SUBSTANCES

(71) Applicants: Valerie Bicard-Benhamou, Darmstadt (DE); Christophe Carola, Bensheim (DE)

(72) Inventors: Valerie Bicard-Benhamou, Darmstadt (DE); Christophe Carola, Bensheim (DE)

(73) Assignee: MERCK PATENT GESELLSCHAFT MIT BESCHRANKTER HAFTUNG, DARMSTADT (DE)

(21) Appl. No.: 13/809,476

(22) PCT Filed: Jun. 24, 2011
(86) PCT No.: PCT/EP2011/003128

§ 371 Date: Jan. 10, 2013

(30) Foreign Application Priority Data
Jul. 10, 2010 (DE) 10 2010 026 775.9

(51) Int. Cl. A61K 8/33; A61Q 17/04

(52) U.S. Cl. CPC A61K 8/33; A61Q 17/04
USPC 424/59

(57) ABSTRACT

The invention relates to the use of alduronic acids and dialdehydes of the aldoses and derivatives thereof as tanning enhancers for dihydroxyacetone or for a mixture of self-tanning substances comprising dihydroxyacetone or as self-tanning substance. The invention furthermore relates to the use thereof for modulation of the hue achieved in the case of tanning with dihydroxyacetone or by the mixture or preparation comprising dihydroxyacetone. The invention furthermore relates to the use thereof as contrast reduction agent in a mixture or preparation comprising at least dihydroxyacetone as self-tanning substance. The use of such compounds enables a significantly improved colouring result of the skin.

The invention relates to the use of at least one compound of the formula I and/or salt thereof as self-tanning substance or as tanning enhancer for dihydroxyacetone or for a mixture of self-tanning substances comprising dihydroxyacetone. The invention furthermore relates to the use of at least one compound of the formula I and/or salt thereof for the modulation of the hue which is achieved on tanning with dihydroxyacetone or by the mixture or preparation comprising dihydroxyacetone. The invention furthermore relates to the use of at least one compound of the formula I and/or salt thereof in a mixture or preparation comprising at least one self-tanning substance as contrast-reduction agent. The invention likewise furthermore relates to a preparation comprising a compound of the formula I and/or salt thereof and at least dihydroxyacetone (DHA) as self-tanning substance and to a process for the preparation of a preparation of this type.

The trend away from refined paleness towards “healthy, sporty brown skin” has been uninterrupted for years. In order to achieve a tanned complexion, people expose their skin to sunlight, since this causes pigmentation due to melanin formation. However, the UV radiation in sunlight also has a damaging effect on the skin. Besides acute damage (sunburn), long-term damage occurs on excessive irradiation with light from the UVB region (wavelength 280-320 nm), such as, for example, an increased risk of contracting skin cancer. Excessive exposure to UVB and UVA radiation (wavelength: 320-400 nm) generates highly reactive free-radical species, which multiply further even after termination of the irradiation, and wrinkling and skin ageing occur as a consequence thereof.

Tanning (pigmentation) of the skin offers natural protection against the adverse consequences of sunlight. The epidermis contains individual pigment-forming cells, the melanocytes, besides the basal cells in its lowest layer, the basal layer. UV light stimulates the production of melanin in these cells, which is transported into the kerantinocytes (horny cells), where it becomes visible as a brown skin colour. Melanin protects the cell nuclei against further irradiation and the adverse effects it causes on the cell DNA. It wraps around the cell nucleus like a parasol and thus protects it against harmful UV radiation.

Depending on the chemical composition of the pigments formed biochemically, a distinction is made between brownish-black eumelanin and reddish-yellow pheomelanin. The skin hue observed is determined by the ratio of these two types of melanin.

This pigment formation starting from the amino acid tyrosine is initiated predominantly by UVB radiation and is known as “indirect pigmentation”. Its development runs over a number of days; the suntan obtained in this way lasts a few weeks. In the case of “direct pigmentation”, which commences with the solar irradiation, predominantly colourless melanin precursors are oxidised by UVA radiation to dark-coloured melanin. Since this oxidation is reversible, it results in skin tanning which only lasts briefly.

Artificial tanning of the skin can be produced externally with the aid of make-up and orally by taking carotenoids.

Much more popular, however, is artificial tanning of the skin which can be achieved by the application of so-called self-tanners.

The substance most frequently employed worldwide for these purposes is 1,3-dihydroxyacetone (DHA), which is used in an amount of 700 t/a. Selftanners can be reacted with the proteins and amino acids of the horny layer of the skin in the sense of a Maillard reaction or via a Michael addition, where polymers which give the skin a brownish hue form via a reaction route which has not yet been clarified completely. This reaction is complete after about 4 to 6 hours. The tan achieved in this way cannot be washed off and is only removed with the normal skin desquamation.

Various substances are already known which are employed as self-tanners and which are described below.

DHA is a water-soluble crystalline solid which is unstable under neutral to basic conditions. This instability is also accompanied by the development of cosmetically undesired malodours.

A problem which can frequently occur on use with self-tanner substances, in particular with 1,3-dihydroxyacetone, is that the tanning of the skin is discoloured towards orange by the dominance of the yellow component.

There also continues to be a demand for dermatologically tolerated skincolouring substances, in particular for combination with dihydroxyacetone, which are suitable for use in cosmetic and/or dermatological preparations or medical devices.

The present invention is concerned with the object of improving the colouring of protein-containing matrices, in particular with respect to a more natural hue.

This problem is solved in accordance with the invention by the subjectmatters of the independent claims. Advantageous embodiments are the subject-matter of the dependent claims.

Surprisingly, it has now been found that compounds of the formula I and/or salts thereof alone are capable of colouring the skin and together with dihydroxyacetone colouring the skin darker than the self-tanner dihydroxyacetone alone and/or together with dihydroxyacetone achieving modulation of the hue to give a more natural hue.

Hereinafter, in each case independently of one another:

Alk stands for a straight-chain or branched C₁- to C₄-alkyl group;
Acy stands for a straight-chain or branched C₁ to C₄-acyl group.

### A:

C₁ to C₄-alkyl group here encompasses methyl, ethyl, propyl-, isopropyl, butyl, x-methylpropyl (x=1;2) and tert-butyl;
C₁ to C₄-acyl group here encompasses formyl-, acetyl, propionyl and 2-methylpropionyl.

The invention accordingly relates to the use of at least one compound of the formula I

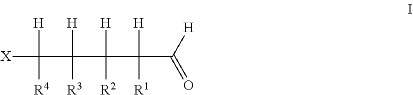

where R¹, R², R³ and R⁴ each stand, independently of one another, for H, OH, O⁻Kt⁺, OAlk, NH₂, NHCOCH₃ or OAcy,
where X stands for COOH or CHO,
where Kt⁺ stands for Na⁺, K⁺, NH₄⁺,
where the C atoms to which R¹, R², R³ or R⁴ are bonded may each, independently of one another, be in the R or S configuration,
where the compounds of the formula I can be in an open-chain form or in a furanoid or pyranoid lactone and/or lactol form,
and/or salt thereof as tanning enhancer for dihydroxyacetone or for a dihydroxyacetone-containing mixture of self-tanning substances.

The invention accordingly furthermore relates to the use of at least one compound of the formula I

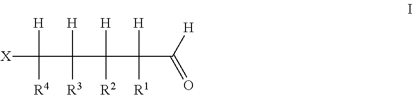

where R¹, R², R³ and R⁴ each stand, independently of one another, for H, OH, O⁻Kt⁺, OAlk, NH₂, NHCOCH₃ or OAcy,
where X stands for COOH or CHO,
where Kt⁺ stands for Na⁺, K⁺, NH₄⁺,
where the C atoms to which R¹, R², R³ or R⁴ are bonded may each, independently of one another, be in the R or S configuration,
where the compounds of the formula I can be in an open-chain form or in a furanoid or pyranoid lactone and/or lactol form,
and/or salt thereof as self-tanning substance.

The invention furthermore relates to the use of at least one compound of the formula I

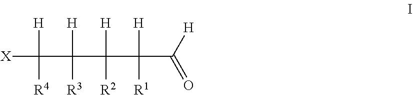

where R¹, R², R³ and R⁴ each stand, independently of one another, for H, OH, O⁻Kt⁺, OAlk, NH₂, NHCOCH₃ or OAcy,
where X stands for COOH or CHO,
where Kt⁺ stands for Na⁺, K⁺, NH₄⁺,
where the C atoms to which R¹, R², R³ or R⁴ are bonded may each, independently of one another, be in the R or S configuration,
where the compounds of the formula I can be in an open-chain form or in a furanoid or pyranoid lactone and/or lactol form,
and/or salt thereof for modulation of the hue achieved in the case of tanning with dihydroxyacetone or by the mixture or preparation comprising dihydroxyacetone.

In the case of a combination of at least one of the compounds of the formula I with dihydroxyacetone for use in cosmetic formulations which serve for colouring the skin, a preferred red shift of the hue achieved is observed.

Thus, with dihydroxyacetone or a mixture of self-tanning substances comprising dihydroxyacetone as self-tanner and on use of at least one compound of the formula I, as described above, self-tanning of the skin can be carried out with a coloration having a natural appearance, without the undesired yellow cast of the coloured skin.

Throughout the document, the term self-tanner or self-tanning substance or self-tanner substance is used synonymously. These terms denote a substance which colours the skin and react with the proteins and/or amino acids of the protein-containing matrix in the sense of a Maillard reaction or via a Michael addition and thus form melanoids. The melanoids are yellow-brown to virtually black organic compounds which can essentially arise through the reaction of carbonyl groups with amino or thio functions. The principle of colouring with formation of melanoids is the basic colouring principle of the self-tanning substances. The colouring capacity of such self-tanning substances can be enhanced through the use of at least one compound of the formula I, as described above.

Dihydroxyacetone, which can be used, inter alia, as self-tanner, colours the skin in accordance with a colouring principle of this type. The use of compounds of the formula I, as described above or described as preferred, enables the colouring process with dihydroxyacetone to be enhanced and/or the hue achieved to be improved. Accordingly, a tanning enhancer is taken to mean a compound of the formula I which is capable, on colouring of the skin with dihydroxyacetone, of achieving an optionally darker hue which is shifted more towards red than a hue which is achieved with dihydroxyacetone or a mixture of self-tanning substances comprising dihydroxyacetone alone.

Furthermore, the use of a tanning enhancer of this type or a compound of the formula I, as described above or described as preferred, on application to the skin reduces the drying-out thereof compared with the use of dihydroxyacetone or a mixture of self-tanning substances comprising dihydroxyacetone alone.

In addition, compounds of the formula I, as described above or described as preferred, can have a contrast-reduction effect, which reduces an uneven skin coloration in use with dihydroxyacetone or a mixture of self-tanning substances comprising dihydroxyacetone and thus reduces the contrast between relatively strongly coloured and less strongly coloured areas of the skin. An uneven skin coloration of this type may arise through uneven pigmentation and/or a different distribution of the horny skin. A contrast-reduction agent is accordingly a substance which reduces an uneven skin coloration by reducing the contrast between relatively strongly coloured and less strongly coloured areas of the skin.

Compounds of the formula I, as described above, in particular glucuronolactone, act as tanning enhancers for dihydroxyacetone, as described above, and at the same time also reduce melanin production, as surprisingly established in cell culture tests with B16V mouse melanoma cells. Compounds of the formula I, as described above, are likewise self-tanning substances, in particular glucuronolactone.

The invention accordingly furthermore relates to the use of at least one compound of the formula I

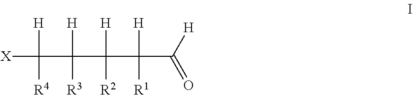

where R¹, R², R³ and R⁴ each stand, independently of one another, for H, OH, O⁻Kt⁺, OAlk, NH₂, NHCOCH₃ or OAcy,
where X stands for COOH or CHO,
where Kt⁺ stands for Na⁺, K⁺, NH₄⁺,
where the C atoms to which R¹, R², R³ or R⁴ are bonded may each, independently of one another, be in the R or S configuration,
where the compounds of the formula I can be in an open-chain form or in a furanoid or pyranoid lactone and/or lactol form,
and/or salt thereof in a mixture or preparation comprising at least one self-tanning substance, as contrast reduction agent.

In particular, the at least one compound of the formula I, as described above, acts in mixtures or preparations comprising at least dihydroxyacetone. The at least one compound of the formula I, the compounds indicated as preferred or particularly preferably glucuronolactone particularly preferably acts in a preparation comprising dihydroxyacetone as self-tanning substance.

A reduction in contrast can therefore be achieved, in particular, by preparations in which combinations according to the invention of dihydroxyacetone or a mixture of self-tanning substances comprising dihydroxyacetone and at least one compound of the formula I, as described above, are additionally combined with a substance which inhibits the biochemical formation of melanin. The combination of tanning mixtures which are based on the Maillard reaction or Michael addition with melanogenesisinhibiting substances has the effect that skin areas which are already hyperpigmented lose their high melanin concentrations and the hue generated by the colorant on the skin surface imposes itself over a large area. Suitable for combination are commercially available melanogenesis inhibitors, such as, for example, ascorbic acid and derivatives thereof, niacinamide, emblica, ellagic acid, mulberry extract, kojic acid, liquorice extract, rucinol, hydroquinone, azelaic acid, arbutin or magnesium ascorbyl phosphate.

The invention therefore likewise relates to a preparation comprising at least one compound of the formula I, as described above, and a melanogenesis inhibitor.

Uneven pigmentation is really not uncommon in the population and is based on different degrees of melanin production by the melanocytes or an irregular distribution of the melanocytes in the skin. Melanin production is triggered by the enzyme tyrosinase, which ultimately sets the colour of the skin, the eyes and the hair.

In combination with dihydroxyacetone, colouring of the skin with more natural skin colorations, in particular shifted into the red colour region, can be achieved, where in addition an advantageous reduction in contrast of unevenly coloured areas of the skin is possible. In addition, in the case of the skin, drying-out of the skin is likewise advantageously reduced by compounds of the formula I.

The substituents R¹, R², R³ and R⁴ are preferably each, independently of one another, H, OH; O⁻Kt⁺, OAlk or OAcy, where Kt⁺, Alk or Acy has one of the meanings mentioned above or below or mentioned as preferred.

A compound of the formula I preferably encompasses derivatives which can be derived from aldoses, but not the aldoses themselves.

Aldoses here are taken to mean monosaccharides containing an aldehyde group and in total six C atoms. The term aldoses thus encompasses the hexoses. Depending on the hexose, the substituents R¹, R², R³ and R⁴ have a corresponding meaning selected from the meanings H, OH, NH₂ or NHCOCH₃, from which the compounds of the formula I, as described above, are then derived. In the case of glucose, for example, the substituents R¹, R², R³ and R⁴ are identical and denote OH. In the case of N-acetylglucosamine, for example, R¹ is NHCOCH₃ and R², R³ and R⁴ are identical and denote OH.

Derivable from the aldoses, the compounds of the formula I thus preferably encompass the alduronic acids and the dialdehydes of the aldoses, and derivatives thereof.

In the case of an alduronic acid, the original aldehyde group of the parent aldose is unchanged, and the CH₂OH group is oxidised to the carboxylic acid. The alduronic acids are named with the word stem of the parent aldose and the ending-urone. The alduronic acid of D-glucose is thus called D-glucuronic acid.

Likewise derivable from the aldoses, the compounds of the formula I thus preferably also encompass dialdehydes, where, starting from the aldose, the CH₂OH group is oxidised to the aldehyde group. These compounds will be referred to below as dialdehyde of the respective aldose.

Furthermore, at least one of the OH groups of the respective aldose derivative in compounds of the formula I, as described above, may be substituted by OAlk, O⁻Kt⁺ or OAcy.

Alk preferably stands for methyl, ethyl, propyl-, isopropyl, or tert-butyl, particularly preferably for methyl, ethyl, or tert-butyl and very particularly preferably for methyl.

Acy preferably stands for formyl-, acetyl, propionyl and butyryl, particularly preferably for formyl-, acetyl and very preferably for acetyl.

Preferably, at least one of the OH groups is substituted by OAlk or OAcy and very particularly preferably all OH groups are unsubstituted.

R¹, R², R³ and R⁴ are particularly preferably OH.

Furthermore, the formula I encompasses not only the open-chain forms, but also cyclic forms. Cyclic forms of the alduronic acids and the dialdehydes of the aldoses and derivatives thereof are, for example, intramolecular monocyclic esters, so-called lactones, monocyclic hemiacetals, so-called lactols, and bicyclic mixed forms thereof, such as, for example, glucoronolactone, in which a γ-lactone may be combined with a cyclic γ-lactol.

The compound of the formula I preferably contains an aldehyde group which is in the form of an intramolecular hemiacetal, the so-called lactol. A lactol is thus taken to mean a cyclic hemiacetal, as usually occurring in the case of monosaccharides.

In the case of a lactol, compounds of the formula I also explicitly encompass α-anomeric and β-anomeric forms thereof and mixtures thereof, where the respective form is determined by the position of the OH group on the anomeric C atom. The anomeric C atom here is the C atom of the original aldehyde group which becomes the anomeric C atom through the formation of the lactol.

X particularly preferably denotes COOH. Particular preference is therefore given to the use in accordance with the invention of at least one compound of the formula I where X═COOH which is very particularly preferably in the form of an intramolecular ester, the so-called lactone.

Preferred compounds of the formula I in which X denotes CHO are the dialdehydes of the following aldoses:

x-allose (x=D,L), x-glucose (x=D,L), x-gulose (x=D,L), x-galactose (x=D,L), x-altrose (x=D,L), x-mannose (x=D,L), x-idose (x=D,L), x-talose (x=D,L), and the corresponding cyclic forms, as described above and below.

Particularly preferred compounds of the formula I in which X denotes COOH are the alduronic acids:

x-alluronic acid (x=D,L), x-glucuronic acid (x=D,L), x-guluronic acid (x=D,L), x-galacturonic acid (x=D,L), x-altruronic acid (x=D,L), x-mannuronic acid (x=D,L), x-iduronic acid (x=D,L), x-taluronic acid (x=D,L), and the corresponding cyclic forms, as described above and below.

The compounds of the formula I which have at least one ring may occur here in a furanoid or pyranoid ring form, analogously to the furanoses or the pyranoses. A furanoid ring form here is to be taken to mean a ring having five carbon atoms in which one carbon atom has been replaced by an oxygen, while a pyranoid ring form is to be taken to mean a ring comprising six carbons in which one carbon atom has been replaced by an oxygen atom.

Lactones can be denoted in accordance with the lactone notation. The αC atom here denotes the C atom following the original carboxyl group, the βC atom denotes the next C atom but one after the original carboxyl group, the γC atom denotes the C atom following the βC atom, and the δC atom denotes the C atom following the γC atom. A γ-lactone thus has a furanoid form, while a δ-lactone has a pyranoid form.

Lactols can be named in accordance with the lactol notation and in a similar manner to the lactone notation. The αC atom here denotes the C atom following the original aldehyde group, the βC atom denotes the next C atom but one after the original aldehyde group, the γC atom denotes the C atom following the βC atom, and the δC atom denotes the C atom following the γC atom. A γ-lactol thus has a furanoid form, while a δ-lactol has a pyranoid form.

The compounds of the formula I, the compounds of the formula I described as preferred or the said dialdehydes of the aldoses or alduronic acids are preferably at least monocyclic, they are particularly preferably monocyclic lactones or bicyclic compounds.

Monocyclic lactones or lactols are preferably δ-lactols and δ-lactones or γ-lactols and γ-lactones; γ-lactols and γ-lactones are particularly preferred, and very particular preference is given to a γ-lactol of a γ-lactone.

x-Glucuronic acid λ-lactone, (x=D,L) x-guluronic acid γ-lactone (x=D,L), x-galacturonic acid γ-lactone (x=D,L), and x-iduronic acid γ-lactone (x=D,L) are thus preferred.

Particular preference is given in accordance with the invention to the use of x-glucuronic acid-γ-lactone (x=D,L), both in the γ-lactone form and also in the bicyclic form containing the γ-lactol of the respective γ-lactone. In the case of a γ-lactol of the respective γ-lactone, an O-bridged ring closure between the aldehyde group and the OH group of the β carbon atom in accordance with the lactone notation is present. Very particular preference is given to the use of glucuronolactone in the bicyclic form.

Advantageous self-tanners which can be employed in a dihydroxyacetone-containing mixture or preparation are, inter alia:

glycerolaldehyde, hydroxymethylglyoxal, γ-dialdehyde, erythrulose, 6-aldo-D-fructose, ninhydrin, 5-hydroxy-1,4-naphtoquinone (juglone) or 2-hydroxy-1,4-naphtoquinone (lawsone) or a mixture of the said compounds. Erythrulose is particularly preferably employed in the dihydroxyacetone-containing mixture.

The at least one compound of the formula I, as described above or described as preferred, can also be used in accordance with the invention together with a mixture of self-tanning substances comprising at least dihydroxyacetone and a further self-tanner selected from the above-mentioned group. By way of example, the mixture to be used in accordance with the invention consists of dihydroxyacetone and at least one further self-tanning substance, as described above. This mixture can then be combined in accordance with the invention with at least one compound of the formula I and employed in cosmetic, dermatological or pharmaceutical preparations, as described below.

Dihydroxyacetone is very particularly preferably employed without further self-tanning substances from the above-mentioned group of self-tanning substances.

The invention furthermore relates to preparations comprising at least one vehicle which is suitable for cosmetic, pharmaceutical, dermatological preparations and at least dihydroxyacetone and at least one compound of the formula I, as described above or described as preferred.

The preparation preferably comprises at least dihydroxyacetone in an amount of 0.01 to 20% by weight, particularly preferably in an amount of 0.5 to 15% by weight and very particularly preferably in an amount of 1 to 8% by weight, based on the total amount of the preparation.

The preparation preferably comprises the at least one compound of the formula I, as described above or described as preferred, in an amount of 0.01 to 20% by weight, particularly preferably in an amount of 0.5 to 15% by weight and very particularly preferably in an amount of 1 to 8% by weight, based on the total amount of the preparation.

The percent by weight ratio of the at least one compound of the formula I to dihydroxyacetone on use as tanning enhancer and/or self-tanning substance and/or colour modulator is 1:20 to 20:1, preferably 1:10 or 10:1, particularly preferably 1:3 to 3:1 and very particularly preferably 1:1.

Preparations having self-tanner properties, in particular those which comprise dihydroxyacetone, tend towards malodours on application to the human skin, which are thought to be caused by degradation products of dihydroxyacetone itself or by products of side reactions and which are regarded as unpleasant by some users. It has been found that these malodours are prevented on use of formaldehyde scavengers and/or flavonoids. The preparation according to the invention comprising at least one compound of the formula I, as described above with the substituents indicated and also preferably mentioned and the individual compounds and at least one self-tanner, can therefore preferably also comprise formaldehyde scavengers and optionally flavonoids for improving the odour. However, the compounds of the formula I claimed for preparations according to the invention, and the corresponding preferred compounds, may also themselves contribute to the improvement in odour.

The formaldehyde scavenger is preferably selected from the group alkali metal, alkaline-earth metal or ammonium disulfite. Particular preference is given to a preparation which comprises, in combination DHA Plus, a mixture of DHA, sodium disulfite and magnesium stearate.

DHA Plus is a product mixture which comprises sodium metabisulfite, synonymous with Na₂S₂O₅ or INCl: sodium disulfite, for the masking, elimination or neutralisation of formaldehyde. The addition of sodium metabisulfite in finished formulations results in significant reduction or suppression of the unpleasant odour. DHA Plus is marketed by Merck, Darmstadt.

The preparation according to the invention comprising at least one compound of the formula I, as described above with the substituents indicated and also preferably mentioned and the compounds mentioned and at least dihydroxyacetone as self-tanner, may particularly preferably comprise flavonoids for improving the odour and optionally for accelerating tanning.

The flavonoid here additionally acts as stabiliser for the self-tanner or the self-tanning substances and/or reduces or prevents or improves storagedependent malodours, which may also arise due to additives or assistants present.

It is preferably a flavonoid in which one or more phenolic hydroxyl groups have been blocked by etherification or esterification. For example, hydroxyethyl-substituted flavonoids, such as, preferably, troxerutin, troxequercetin, troxeisoquercetin or troxeluteolin, and flavonoid sulfates or flavonoid phosphates, such as, preferably, rutin sulfates, have proven to be particularly highly suitable flavonoids here. Particular preference is given in the sense of the use according to the invention to rutin sulfate and troxerutin. Very particular preference is given to the use of troxerutin.

The preferred flavonoids have a non-positively charged flavan skeleton. It is thought that metal ions, such as, for example, Fe²⁺/Cu²⁺, are complexed by these flavonoids and autooxidation processes in the case of fragrances or compounds whose degradation results in malodours are thus prevented or reduced.

Particular preference is given to a preparation which, besides at least one compound of the formula I, comprises DHA Rapid. DHA Rapid is a product mixture comprising dihydroxyacetone and troxerutin, from Merck, Darmstadt. This particularly preferred preparation may optionally also comprise a formaldehyde scavenger, for example sodium disulfite.

Corresponding premixes and preparations which comprise formaldehyde scavengers and optionally flavonoids in order to improve the odour on the skin are described in the German patent application DE 10 2007 013 368 A1, the contents of which in this respect expressly also belong to the disclosure content of the present application.

The preparations here are usually preparations which can be applied topically, for example cosmetic or dermatological formulations or medical devices. In this case, the preparations comprise a cosmetically or dermatologically suitable vehicle and, depending on the desired property profile, optionally further suitable ingredients. In the case of pharmaceutical preparations, the preparations in this case comprise a pharmaceutically tolerated vehicle and optionally further pharmaceutical active compounds.

“Can be applied topically” in the sense of the invention means that the preparation is applied externally and locally, i.e. that the preparation must be suitable for, for example, application to the skin.

In the sense of the present invention, the term agent or formulation is also used synonymously alongside the term preparation.

The preparations may include or comprise, essentially consist of or consist of the said requisite or optional constituents. All compounds or components which can be used in the preparations are either known and commercially available or can be synthesised by known processes.

Further preferred combinations of embodiments are disclosed in the claims.

The invention also relates to a process for the preparation of a preparation, as described above, in which at least one compound of the formula I as described above or described as preferred, is mixed with a vehicle and optionally with further active compounds or assistants. At least one further self-tanner substance is optionally then added and mixed, and finally dihydroxyacetone is added and mixed. Suitable vehicles and active compounds or assistants are described in detail in the following part.

In the preparations described, which, in accordance with the invention, comprise at least one compound of the formula I, as described above or described as preferred, and dihydroxyacetone, coloured pigments may furthermore also be present, where the layer structure of the pigments is not limited.

The coloured pigment should preferably be skin-coloured or brownish on use of 0.5 to 5% by weight. The choice of a corresponding pigment is familiar to the person skilled in the art.

Besides the compounds of the formula I, at least dihydroxyacetone as self-tanning substance and the optional other ingredients, preferred preparations may comprise further organic UV filters, so-called hydrophilic or lipophilic sun-protection filters, which are effective in the UVA region and/or UVB region and (/or IR and/or VIS region (absorbers). These substances can be selected, in particular, from cinnamic acid derivatives, salicylic acid derivatives, camphor derivatives, triazine derivatives, β,β-diphenylacrylate derivatives, p-aminobenzoic acid derivatives and polymeric filters and silicone filters, which are described in the application WO-93/04665. Further examples of organic filters are indicated in the patent application EP-A 0 487 404. The said UV filters are usually named below in accordance with INCl nomenclature.

Particularly suitable for a combination are:

para-aminobenzoic acid and derivatives thereof: PABA, Ethyl PABA, Ethyl dihydroxypropyl PABA, Ethylhexyl dimethyl PABA, for example marketed by ISP under the name “Escalol 507”, Glyceryl PABA, PEG-25 PABA, for example marketed under the name “Uvinul P25” by BASF.

Salicylates: Homosalate marketed by Merck under the name “Eusolex HMS”; Ethylhexyl salicylate, for example marketed by Symrise under the name “Neo Heliopan OS”, Dipropylene glycol salicylate, for example marketed by Scher under the name “Dipsal”, TEA salicylate, for example marketed by Symrise under the name “Neo Heliopan TS”.

β,β-Diphenylacrylate derivatives: Octocrylene, for example marketed by Merck under the name “Eusolex® OCR”, “Uvinul N539” from BASF, Etocrylene, for example marketed by BASF under the name “Uvinul N35”.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethylamino-2-hydroxybenzoyl)benzoate or 2-hydroxy-4-methoxybenzophenone, marketed by Merck, Darmstadt, under the name Eusolex® 4360.

Benzylidenecamphor derivatives: 3-Benzylidenecamphor, for example marketed by Chimex under the name “Mexoryl SD”, 4-Methylbenzylidenecamphor, for example marketed by Merck under the name “Eusolex 6300”, benzylidenecamphorsulfonic acid, for example marketed by Chimex under the name “Mexoryl SL”, Camphor benzalkonium methosulfate, for example marketed by Chimex under the name “Mexoryl SO”, terephthalylidenedicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyl dibenzimidazole tetrasulfonate, for example marketed by Symrise under the name “Neo Heliopan AP”.

Phenylbenzotriazole derivatives: Drometrizole trisiloxane, for example marketed by Rhodia Chimie under the name “Silatrizole”, Methylenebis(benzotriazolyl)tetramethylbutylphenol in solid form, for example marketed by Fairmount Chemical under the name “MIXXIM BB/100”, or in micronised form as an aqueous dispersion, for example marketed by BASF under the name “Tinosorb M”.

Triazine derivatives: ethylhexyltriazone, for example marketed under the name “Uvinul T150” by BASF, diethylhexylbutamidotriazone, for example marketed under the name “Uvasorb HEB” by Sigma 3V, 2,4,6-tris(diisobutyl 4′-aminobenzalmalonate)-s-triazine or 2,4,6-tris(biphenyl)-1,3,5-triazine. marketed as Tinosorb A2B by BASF, 2,2′-[6-(4-methoxyphenyl)-1,3,5-triazine-2,4-diyl]bis[5-(2-ethylhexyl)oxy]phenol, marketed as Tinosorb S by BASF, N2,N4-bis[4-[5-(1,1-dimethylpropyl)-2-benzoxazolyl]phenyl]-N-6-(2-ethylhexyl)-1,3,5-triazine-2,4,6-triamine marketed as Uvasorb K 2A by Sigma 3V.

Anthraniline derivatives: Menthyl anthranilate, for example marketed by Symrise under the name “Neo Heliopan MA”.

Imidazole derivatives: Ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl) imino]-6-(2-ethylhexyl)imino-1,3,5-triazine, for example marketed by Sigma 3V under the name Uvasorb K2A, and mixtures comprising this.

Piperazine derivatives, such as, for example, the compound

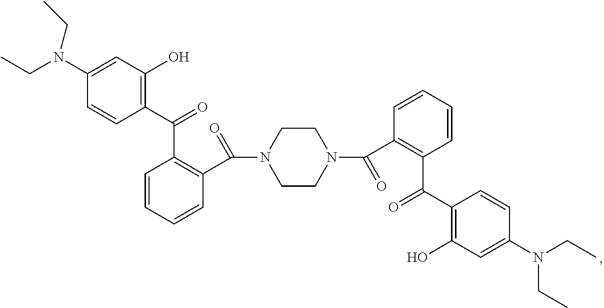 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

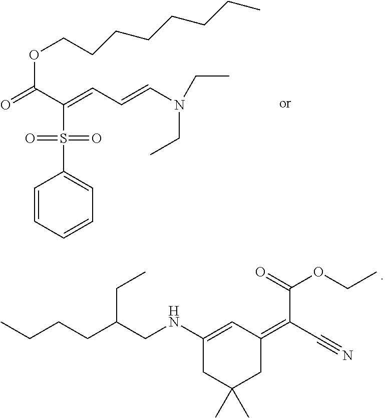

It is also possible to use UV filters based on polysiloxane copolymers having a random distribution in accordance with the following formula, where, for example, a=1.2; b=58 and c=2.8:

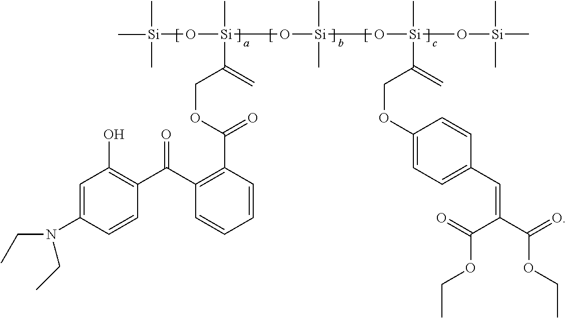

The compounds listed should only be regarded as examples. It is of course also possible to use other UV filters.

Suitable organic UV-protecting substances can preferably be selected from the following list: Ethylhexyl salicylate, Phenylbenzimidazolesulfonic acid, Benzophenone-3, Benzophenone-4, Benzophenone-5, n-Hexyl 2-(4-diethylamino-2-hydroxybenzoyl)benzoate, 4-Methylbenzylidenecamphor, Terephthalylidenedicamphorsulfonic acid, Disodium phenyldibenzimidazoletetrasulfonate, Methylenebis(benzotriazolyl)tetramethylbutylphenol, Ethylhexyl Triazone, Diethylhexyl Butamido Triazone, Drometrizole trisiloxane, Polysilicone-15,1,1-Dicarboxy(2,2′-dimethylpropyl)-4,4-diphenylbutadiene, 2,4-bis[5-1 (dimethylpropyl)benzoxazol-2-yl(4-phenyl) imino]-6-(2-ethylhexyl)imino-1,3,5-triazine and mixtures thereof.

These organic UV filters are generally incorporated into formulations in an amount of 0.01 percent by weight to 20 percent by weight, preferably 1% by weight-10% by weight.

Besides the compounds of the formula I, at least dihydroxyacetone as self-tanning substance and the optional other ingredients, as described above, the preparations may comprise further inorganic UV filters, so-called particulate UV filters.

These combinations with particulate UV filters are possible both as powder and also as dispersion or paste of the following types.

Preference is given here both to those from the group of the titanium dioxides, such as, for example, coated titanium dioxide (for example Eusolex®T-2000, Eusolex® T-AQUA, Eusolex® T-AVO, Eusolex® T-OLEO), zinc oxides (for example Sachtotec), iron oxides or also cerium oxides and/or zirconium oxides.

Furthermore, combinations with pigmentary titanium dioxide or zinc oxide are also possible, where the particle size of these pigments are greater than or equal to 200 nm, for example Hombitan® FG or Hombitan® FFPharma.

It may furthermore be preferred for the preparations to comprise inorganic UV filters which have been aftertreated by conventional methods, as described, for example, in Cosmetics & Toiletries, 1990, 105, 53-64. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerine.

Particulate UV filters which are preferably employed here are:
untreated titanium dioxides, such as, for example, the products Microtitanium Dioxide MT 500 B from Tayca; titanium dioxide P25 from Degussa,
aftertreated micronised titanium dioxides with aluminium oxide and silicon dioxide aftertreatment, such as, for example, the product “Microtitanium Dioxide MT 100 SA from Tayca; or the product “Tioveil Fin” from Uniqema,
aftertreated micronised titanium dioxides with aluminium oxide and/or aluminium stearate/laurate aftertreatment, such as, for example, Microtitanium Dioxide MT 100 T from Tayca, Eusolex T-2000 from Merck,
aftertreated micronised titanium dioxides with iron oxide and/or iron stearate aftertreatment, such as, for example, the product “Microtitanium Dioxide MT 100 F” from Tayca,
aftertreated micronised titanium dioxides with silicon dioxide, aluminium oxide and silicone aftertreatment, such as, for example, the product “Microtitanium Dioxide MT 100 SAS”, from Tayca,
aftertreated micronised titanium dioxides with sodium hexametaphosphates, such as, for example, the product “Microtitanium Dioxide MT 150 W” from Tayca.

The treated micronised titanium dioxides employed for the combination may also be aftertreated with:
octyltrimethoxysilanes; such as, for example, the product Tego Sun T 805 from Evonik Goldschmidt GmbH,
silicon dioxide; such as, for example, the product Parsol T-X from DSM,
aluminium oxide and stearic acid; such as, for example, the product UV-Titan M160 from Sachtleben,
aluminium and glycerine; such as, for example, the product UV-Titan from Sachtleben,
aluminium and silicone oils, such as, for example, the product UV-Titan M262 from Sachtleben,
sodium hexametaphosphate and polyvinylpyrrolidone,
polydimethylsiloxanes, such as, for example, the product 70250 Cardre UF TiO2SI3″ from Cardre,
polydimethylhydrogenosiloxanes, such as, for example, the product Microtitanium Dioxide USP Grade Hydrophobic” from Color Techniques.

The combination with the following products may furthermore also be advantageous:
untreated zinc oxides, such as, for example, the product Z-Cote from BASF (Sunsmart), Nanox from Elementis
aftertreated zinc oxides, such as, for example, the following products:
   “Zinc Oxide CS-5” from Toshibi (ZnO aftertreated with polymethylhydrogenosiloxanes)
   Nanogard Zinc Oxide FN from Nanophase Technologies
   “SPD-Z1” from Shin-Etsu (ZnO aftertreated with a silicone-grafted acrylic polymer, dispersed in cyclodimethylsiloxanes
   “Escalol Z100” from ISP (aluminium oxide-aftertreated ZnO dispersed in an ethylhexyl methoxycinnamate/PVP-hexadecene/methicone copolymer mixture)
   “Fuji ZNO-SMS-10” from Fuji Pigment (ZnO aftertreated with silicon dioxide and polymethylsilesquioxane);
   untreated cerium oxide micropigment, for example with the name “Colloidal Cerium Oxide” from Rhone Poulenc untreated and/or aftertreated iron oxides with the name Nanogar from Arnaud.

By way of example, it is also possible to employ mixtures of various metal oxides, such as, for example, titanium dioxide and cerium oxide, with and without aftertreatment, such as, for example, the product Sunveil A from Ikeda. In addition, mixtures of aluminium oxide, silicon dioxide and silicone-aftertreated titanium dioxide. zinc oxide mixtures, such as, for example, the product UV-Titan M261 from Sachtleben, can also be used in combination with the UV protection agents according to the invention.

These inorganic UV filters are generally incorporated into the preparations in an amount of 0.1 percent by weight to 25 percent by weight, preferably 2% by weight-10% by weight.

By combination of one or more of the said compounds having a UV filter action, the protective action against harmful effects of the UV radiation can be optimised.

All said UV filters can also be employed in encapsulated form. In particular, it is advantageous to employ organic UV filters in encapsulated form.

The capsules in preparations to be employed in accordance with the invention are preferably present in amounts which ensure that the encapsulated UV filters are present in the preparation in the percent by weight ratios indicated above.

Preferred preparations may also comprise at least one further cosmetic active compound, for example selected from antioxidants, anti-ageing active compounds, anti-wrinkle, anti-flake, anti-acne, deodorants, anti-cellulite active compounds, self-tanning substances, skin-lightening active compounds or vitamins.

The protective action of preparations against oxidative stress or against the effect of free radicals can be improved if the preparations comprise one or more antioxidants, the person skilled in the art being presented with absolutely no difficulties in selecting antioxidants which act suitably quickly or with a time delay.

There are many proven substances known from the specialist literature which can be used as antioxidants, for example amino acids (for example glycine, histidine, tyrosine, tryptophan) and derivatives thereof, imidazoles, (for ecample urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmmol/kg), and also (metal) chelating agents, (for example α-hydroxyfatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate) and coniferyl benzoate of benzoin resin, rutinic acid and derivatives thereof, a-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the formulae A or B

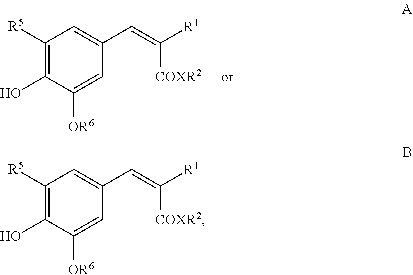

in which
R¹ can be selected from the group —C(O)CH₃, —CO₂R³, —C(O)NH₂ and —C(O)N(R⁴)₂,
X denotes O or NH,′
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes H or linear or branched alkyl having 1 to 8 C atoms or linear or branched alkoxy having 1 to 8 C atoms and
R⁶ denotes linear or branched alkyl having 1 to 8 C atoms, preferably derivatives of 2-(4-hydroxy-3,5-dimethoxybenzylidene)malonic acid and/or 2-(4-hydroxy-3,5-dimethoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-dimethoxybenzyl)malonate (for example RonaCare® AP).

Mixtures of antioxidants are likewise suitable for use in the cosmetic preparations according to the invention. Known and commercial mixtures are, for example, mixtures comprising, as active ingredients, lecithin, L-(+)-ascorbyl palmitate and citric acid, natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed in such preparations with the compounds according to the invention in percent by weight ratios in the range from 1000:1 to 1:1000, preferably in percent by weight ratios of 100:1 to 1:100.

Of the phenols, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers and I. M. C. M. Rietjens (Free Radical Biology&Medicine 2001, 31(7), 869-881, have investigated the pH dependence of the antioxidant action of hydroxyflavones. Quercetin exhibits the highest activity amongst the structures investigated over the entire pH range.

Suitable anti-ageing active compounds, in particular for skin-care preparations, are preferably so-called compatible solutes. These are substances which are involved in the osmosis regulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and respective precursors thereof. Osmolytes in the sense of German patent application DE-A-10133202 are taken to mean, in particular, substances from the group of the polyols, such as, for example, myo-inositol, mannitol or sorbitol, and/or one or more of the osmolytically active substances mentioned below: taurine, choline, betaine, phosphorylcholine, glycerophosphorylcholines, glutamine, glycine, α-alanine, glutamate, aspartate, proline and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidinecarboxylic acids (such as ectoin and hydroxyectoin), proline, betaine, glutamine, cyclic diphosphoglycerate, N.-acetylornithine, trimethylamine N-oxide di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) or/and dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid) and derivatives thereof.

Additionally, anti-aging active compounds which can be used are products from Merck, such as, for example, 5,7-dihydroxy-2-methylchromone, marketed under the trade name RonaCare®Luremine, or the commercial products RonaCare®Isoquercetin, RonaCare®Tilirosid or RonaCare® Cyclopeptide 5.

The preparations may also comprise one or more further skin-lightening active compounds or synonymously depigmentation active compounds. Skin-lightening active compounds can in principle be all active compounds known to the person skilled in the art. Examples of compounds having skin-lightening activity are hydroquinone, kojic acid, arbutin, aloesin, niacinamide, azelaic acid, elagic acid, mulberry extract, magnesium ascorbyl phosphate, liquorice extract, emblica, ascorbic acid or rucinol.

The preparations to be employed may comprise vitamins as further ingredients. Preference is given to vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. In the case of cosmetic application, vitamins are usually added with the flavonoid-containing premixes or preparations in ranges from 0.01 to 5.0% by weight, based on the total weight.

Nutrition-physiological applications are oriented towards the respective recommended vitamin requirement.

The retinoids described are at the same time also effective anti-cellulite active compounds. A likewise known anti-cellulite active compound is caffeine.

The said constituents of the preparation can be incorporated in the usual manner, with the aid of techniques which are well known to the person skilled in the art.

Suitable preparations are those for external application, for example can be sprayed onto the skin as cream or milk (O/W, W/O, O/W/O, W/O/W), as lotion or emulsion, in the form of oily-alcoholic, oily-aqueous or aqueous-alcoholic gels or solutions. They can be in the form of solid sticks or formulated as an aerosol.

The following may preferably be mentioned as application form of the preparations to be employed: solutions, suspensions, emulsions, PIT emulsions, pastes, ointments, gels, creams, lotions, powders, soaps, surfactant-containing cleansing preparations, oils, aerosols plasters, compresses, bandages and sprays.

Preferred assistants originate from the group of preservatives, stabilisers, solubilisers, colorants, odour improvers.

Ointments, pastes, creams and gels may comprise the customary vehicles which are suitable for topical application, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example chlorofluorocarbons, propane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

A preferred solubiliser in general is 2-isopropyl-5-methylcyclohexanecarbonyl-D-alanine methyl ester.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, powder make-up, emulsion make-up and wax make-up, and sunscreen, pre-sun and after-sun preparations.

The preferred preparation forms also include, in particular, emulsions.

Emulsions are advantageous and comprise, for example, the said fats, oils, waxes and other fatty substances, as well as water and an emulsifier, as usually used for a preparation of this type.

The lipid phase may advantageously be selected from the following group of substances:
mineral oils, mineral waxes
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of esters of aromatic carboxylic acid and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group branched and unbranched hydrocarbons and hydrocarbon waxes, silicone oils, dialkyl ethers, the group of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may, for example, advantageously be selected from the group of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as sole lipid component of the oil phase.

The aqueous phase of the preparations to be employed optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

Emulsions are advantageous and comprise, for example, the said fats, oils, waxes and other fatty substances, as well as water and an emulsifier, as usually used for a formulation of this type.

In a preferred embodiment, the preparations to be employed comprise hydrophilic surfactants. The hydrophilic surfactants are preferably selected from the group of the alkylglucosides, acyl lactylates, betaines and coconut amphoacetates.

It is likewise advantageous to employ natural or synthetic raw materials and assistants or mixtures which are distinguished by an effective content of the active compounds used in accordance with the invention, for example Plantaren® 1200 (Henkel KGaA), Oramix® NS 10 (Seppic).

The cosmetic and dermatological preparations may exist in various forms. Thus, they may be, for example, a solution, a water-free preparation, an emulsion or microemulsion of the water-in-oil (W/O) type or of the oil-inwater (O/W) type, a multiple emulsion, for example of the water-in-oil-inwater (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatine, wax matrices or liposomally encapsulated. In particular, wax matrices, as described in DE-A-43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions.

The co-emulsifiers selected are advantageously, for example, O/W emulsifiers, principally from the group of substances having HLB values of 11-16, very particularly advantageously having HLB values of 14.5-15.5, so long as the O/W emulsifiers have saturated radicals R and R′. If the O/W emulsifiers have unsaturated radicals R and/or R′, or if isoalkyl derivatives are present, the preferred HLB value of such emulsifiers may also be lower or higher.

It is advantageous to select the fatty alcohol ethoxylates from the group of the ethoxylated stearyl alchols, cetyl alcohols, cetylstearyl alcohols (cetearyl alcohols).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laureth1-4 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/cprinate, polyethylene glycol (20) glyceryl oleate, polyethylene glycol (20) glyceryl isostearate, polyethylene glycol (18) glyceryl oleate (cocoate).

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention:

fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG-30 dipolyhydroxystearate.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, dyes and/or pigments, and other ingredients usually used in cosmetics.

The dispersant or solubiliser used can be an oil, wax or other fatty bodies, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion which is in the form of a protective cream or milk and comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The preparation may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a preparation is formulated as an aerosol, use is generally made of the customary propellants, such as alkanes, air, nitrogen, dinitrogen monoxide, preferably alkanes.

Even without further comments, it is assumed that a person skilled in the art will be able to utilise the above description in the broadest scope. The preferred embodiments and examples should therefore merely be regarded as descriptive disclosure which is absolutely not limiting in any way. The complete disclosure content of all applications and publications mentioned above and below is incorporated into this application by way of reference. The percent by weight ratios of the individual ingredients in the preparations of the examples expressly belong to the disclosure content of the description and can therefore be utilised as features.

Further important features and advantages of the invention arise from the sub-claims and from the examples.

It goes without saying that the features mentioned above and still to be explained below can be used not only in the respective combination indicated, but also in other combinations or in isolation without leaving the framework of the present invention.

Preferred embodiments of the invention are described in the examples and are explained in greater detail in the following description without restricting the scope of the present invention.

### EXAMPLE 1

### “In Vivo” Study

Four participants (female and male) were selected for the study. In order to carry out the study, test fields measuring 4×4.5 cm² were marked on both forearms of each participant.

The following test formulation was used:

| | | Wt. |
|---|---|---|
| Constituents/trade name | INCI | [%] |
| Pioneer Gold (Hansen & Rosenthal | Paraffin oil, isopropyl | 18.0 |
| KG) | palmitate, polyglycerol | |
| | ester | |
| Pioneer NP37 G (Hansen & | Sodium carboxyl vinyl | 1.5 |
| Rosenthal KG) | polymer | |
| Water (Milipore plant A25) | Water | 69.7 |
| Dihydroxyacetone (Merck) | Dihydroxyacetone | 5.0 |
| Glucuronolactone | Glucuronolactone | 5.0 |
| Pioneer GCK (Hansen & Rosenthal | N-Cocoyl glutamic acid | 0.8 |
| KG) | monosodium salt | |

For comparison, the above-mentioned test formulation was used with 6 percent by weight of dihydroxyacetone without addition of glucuronolactone. Glucuronolactone was employed in the bicyclic form.

One test field was treated with 2 μl/cm² of the O/W formulation described above comprising 6 percent by weight of DHA, the other test field was treated with 2 μl/cm² of the O/W formulation described above comprising 5 percent by weight of DHA and 5 percent by weight of glucuronolactone (the percent by weight ratio is accordingly 1:1). For this purpose, the sample was applied in a grid, so that the test amount is distributed uniformly on the test field. Using a glove finger, the sample was rubbed uniformly with gentle pressure, so that the entire test area was treated and the preparation was absorbed (about 30 seconds). In each case, one treatment was carried out per forearm. A colour measurement was carried out in the test field before treatment and 24 h after the treatment. The colour measurements were carried out using a Minolta CR-300 Chroma-meter, and the L, a and b values in accordance with the CIE (Commission Internationale de l'Eclairage) definition were determined. The decrease in luminance and the increase in the colour having a red and yellow wavelength were determined compared with the colour of the skin before the treatment. The following values were obtained:

| | DHA | DHA:glucuronolactone |
|---|---|---|
| | (6 wt. %) | (5 wt. %:5 wt. %) |
| ΔL | −3.62 | −5.08 |
| Δa | 0.97 | 2.63 |
| Δb | 4.79 | 4.69 |

### EXAMPLE 2

### Contrast-Reducing Effect

Description of the performance of the B16 V mouse melanoma cell test:

B16V mouse melanoma cells (manufacturer: DSMZ; Article No,: ACC370) in RPMI medium (Invitrogen, Article No.: 31870), to which 10% of FBS (foetal bovine serum, Invitrogen, Article No: 10499044), 2 mM L-glutamine (Invitrogen, Article No: 25030) and 1 mM sodium pyruvate (Invitrogen, Article No:11360) had additionally been added (modified RPMI medium), are transferred into a culture bottle and incubated at 37° C. and 5% CO₂ for 72 h. The medium is separated off, and the cells are washed with 10 ml of D-PBS (Invitrogen, Article No. 14190) and subsequently filtered off with suction. 1 ml of HyQtase cell detachment solution (Hyclone, Article No. SV30030.01) is added to the cells. The bottle is swirled a number of times, and the HyQtase cell detachment solution is subsequently removed by suction. The cells are then incubated in the incubator at 37° C. and 5% CO₂ for 5 min. The cells are taken up in the modified RPMI medium (Invitrogen, Article No.: 31870), and the cell count is determined. To this end, the cells are stained with Tryptan Blue and counted in a Neubauer chamber. The cells are subsequently sown out again in the modified RPMI medium in a defined cell count of 80,000 cells per well (6-well clear plate, TCT, PS (Nunc)).

The plate is subsequently incubated at 37° C. and 5% CO₂ for 24 h, the medium is then removed. 1980 μl of the glucuronolactone dilution are subsequently added. For this substance dilution, glucuronolactone is dissolved in DMSO and subsequently filtered through a sterile filter (0.2 μm, Millipore, Article No. SLLG013SL). The solution is then diluted with the modified RPMI medium (in this case the FBS content in the RPMI medium is only 5%) in such a way that the glucuronolactone concentration is 10 mM. 20 μl of an alpha-MSH solution in DMSO (alpha-melanocyte-stimulating hormone=alpha-MSH) (DMSO, Sigma, Article No.:D2650) are then added, so that the alpha-MSH concentration in the well is 10⁻⁸ M. The plate is subsequently incubated again at 37° C. and 5% CO₂ for 24 h. The process described in this section is repeated a further twice in total.

After the final incubation period, the medium is removed by suction, and the cells are washed with 1000 μl of D-PBS (Invitrogen, Article No. 14190). The medium is again removed by suction 250 μl of HyQtase cell detachment solution (Hyclone, Article No. SV30030.01) are added to the cells. The 6-well plate is swirled a number of times, and the HyQtase cell detachment solution is subsequently removed by suction. The cells are then incubated in the incubator at 37° C. and 5% CO₂ for 5 min. The cells are then taken up in 1.5 ml of DPBS (Invitrogen, Article No. 14190) and trans-ferred into a cup (SARSTEDT, Ref. 72.692.005). The cell count is subsequently determined. To this end, the cells are stained with Tryptan Blue and counted in a Neubauer chamber. The cells are then centrifuged for 1 min at 3500 g. The pellets obtained are photographed, and the supernatant is subsequently removed by suction. The pellets are dissolved in 1 ml of 1N NaOH at 80° C. for 1 h and then cooled to RT. 200 μl are then pipetted as quadruple determination (per cup) into a 96-well plate (VWR, Article No. 4100636981), and the absorption at a wavelength of 405 nm is determined. (Safire, Tecan). The content of melanin can be determined in this way by means of a calibration line.

The following table shows the results of the determination of the melanin concentration obtained for the substance dilutions indicated. The other values were determined relative to the blank sample with dimethyl sulfoxide (DMSO) and a-MSH, whose measurement was set at 100%

| Substance dilution | Melanin concentration % |
|---|---|
| Glucuronolactone (10 mM) | 56.7 |
| DMSO (0.1%) + α-MSH (10⁻⁸ M) | 100 |
| Glucuronolactone (10 mM) + α-MSH | 46.9 |

### EXAMPLE 3

### Ex-Vivo Study

### Preparation of the Explants

12 explants from the stomach tissue of a 49-year-old pale-skinned woman (Caucasian type) having an average diameter of 10 mm are prepared (reference: P831-AB49).

The explants are kept in a BEM medium (B10-EC's explants medium) at 37° C. in a moist atmosphere comprising 5% of CO₂.

The following samples are tested: (in each case 3 explants), where the solutions of the corresponding substances used comprise 90% of water and 10% of ethanol.

| | |
|---|---|
| A negative control (untreated sample) | → T0 |
| A positive control (dihydroxyacetone (DHA) 5%) | → R |
| Glucuronolactone (1%) + DHA (5%) | → P1 |
| Glucuronolactone (1%) | → P2 |

Glucuronolactone is employed in the bicyclic form.

2 μl of the respective sample are added to the explants and distributed for the first time (D0), 1 day later (D1), 2 days later (D2), 3 days later (D3) and 4 days later (D4). The colour of the explants is determined chromametrically. The measurements are carried out on addition (D0), on day 1 (D1), on day 5 (D5) and on day 11 (D11).

The colour measurements are carried out using a Minolta CR-300 Chroma-meter, and the L, a and b values are read off correspondingly on the instrument.

### Principle of the Chromametric Measurement:

The following parameters are set:

In 1931, a test series was carried out by the CIE (Commision Internationale de L′Eclairage), where the “2-degree standard observer” was defined. A colour area is present here which is viewed at a viewing angle of 2 degrees. This viewing angle is used as standard in the chromametric measurement.
Irradiation source D65, corresponding to daylight
Region of the measurement: 3 mm
Parameters used: L*a*b*

The sample is placed on the bearing rail with the epidermis facing downwards. The irradiation is carried out from the underside through the area indicated (3 mm).

### Contrast Study:

In the colorimetric determinations <<L a b>>, the L value determines the contrast of the skin. If the L value drops, the skin appears darker.

### Results:

Table 1 shows the measurement values of the explants for samples T0, R, P1 and P2 (in each case n=3).

| Measurement | L | a | b | ΔL | Δa | Δb |
|---|---|---|---|---|---|---|
| T01 D0 | 63.18 | 2.58 | 16.54 | — | — | — |
| T01 D1 | 64.58 | 2.87 | 16.29 | 1.39 | 0.29 | −0.25 |
| T01 D5 | 65.76 | 2.38 | 14.99 | 2.58 | −0.2 | −1.56 |
| T01 D11 | 66.67 | 2.19 | 13.78 | 3.48 | −0.39 | −2.76 |
| T02 D0 | 63.37 | 1.79 | 17.92 | — | — | — |
| T02 D1 | 64.01 | 1.78 | 17.38 | 0.65 | −0.01 | −0.54 |
| T02 D5 | 64.67 | 1.64 | 13.95 | 1.3 | −0.15 | −3.97 |
| T02 D11 | 63.9 | 1.33 | 12.18 | 0.53 | −0.46 | −5.74 |
| T03 D0 | 62.61 | 2.76 | 19.21 | — | — | — |
| T03 D1 | 65.58 | 3.13 | 18.32 | 2.98 | 0.37 | −0.89 |
| T03 D5 | 64.79 | 2.87 | 17.3 | 2.18 | 0.11 | −1.9 |
| T03 D11 | 64.32 | 3.21 | 15 | 1.72 | 0.45 | −4.21 |
| R1 D0 | 60.86 | 4.79 | 20.01 | — | — | — |
| R1 D1 | 60.28 | 4.7 | 17.83 | −0.58 | −0.09 | −2.18 |
| R1 D5 | 60.66 | 3.03 | 18.2 | −0.2 | −1.76 | −1.81 |
| R1 D11 | 61.43 | 3.42 | 17.18 | 0.57 | −1.37 | −2.83 |
| R2 D0 | 58.53 | 5.79 | 20.21 | — | — | — |
| R2 D1 | 58.94 | 4.53 | 17.76 | 0.41 | −1.26 | −2.45 |
| R2 D5 | 61.5 | 3.21 | 16.72 | 2.97 | −2.58 | −3.49 |
| R2 D11 | 58.09 | 3.46 | 16.35 | −0.44 | −2.33 | −3.86 |
| R3 D0 | 58.01 | 6.13 | 19.96 | — | — | — |
| R3 D1 | 58.33 | 5.49 | 16.94 | 0.32 | −0.64 | −3.02 |
| R3 D5 | 59.23 | 4.58 | 17.97 | 1.22 | −1.55 | −1.99 |
| R3 D11 | 58.81 | 2.24 | 15.47 | 0.8 | −3.89 | −4.49 |
| P1 1 D0 | 62.07 | 2.12 | 18.37 | — | — | — |
| P1 1 D1 | 65.87 | 1.36 | 17.1 | 3.8 | −0.76 | −1.27 |
| P1 1 D5 | 64.18 | 1.19 | 16.69 | 2.11 | −0.93 | −1.68 |
| P1 1 D11 | 64.3 | 2.51 | 16.37 | 2.23 | 0.39 | −2.01 |
| P1 2 D0 | 63.51 | 2.2 | 16.95 | — | — | — |
| P1 2 D1 | 66.8 | 2.34 | 16.61 | 3.29 | 0.13 | −0.34 |
| P1 2 D5 | 63.27 | 1.63 | 17.22 | −0.24 | −0.57 | 0.27 |
| P1 2 D11 | 64.37 | 2 | 16.96 | 0.87 | −0.2 | 0.01 |
| P1 3 D0 | 62.51 | 2.73 | 17.34 | — | — | — |
| P1 3 D1 | 65.76 | 2.12 | 16.15 | 3.25 | −0.61 | −1.2 |
| P1 3 D5 | 65.31 | 2.78 | 16.48 | 2.8 | 0.05 | −0.86 |
| P1 3 D11 | 64.65 | 1.77 | 16.86 | 2.14 | −0.96 | −0.48 |
| P2 1 D0 | 64.07 | 0.87 | 17.1 | — | — | — |
| P2 1 D1 | 65.12 | 1.32 | 17.81 | 1.05 | 0.46 | 0.71 |
| P2 1 D5 | 62.73 | 1.11 | 16.51 | −1.33 | 0.25 | −0.59 |
| P2 1 D11 | 64.22 | 1.87 | 14.34 | 0.16 | 1 | −2.76 |
| P2 2 D0 | 62.49 | 2.56 | 17.34 | — | — | — |
| P2 2 D1 | 64.59 | 2.52 | 17.14 | 2.1 | −0.04 | −0.2 |
| P2 2 D5 | 63.62 | 2.78 | 16.88 | 1.12 | 0.22 | −0.46 |
| P2 2 D11 | 65.7 | 2.44 | 15.04 | 3.2 | −0.12 | −2.3 |
| P2 3 D0 | 62.34 | 2.28 | 16.15 | — | — | — |
| P2 3 D1 | 62.71 | 2.83 | 16.29 | 0.37 | 0.55 | 0.13 |
| P2 3 D5 | 63.56 | 2.54 | 15.95 | 1.22 | 0.26 | −0.2 |
| P2 3 D11 | 62.69 | 2.65 | 15.57 | 0.35 | 0.38 | −0.59 |

In comparison with the untreated sample, glucuronolactone exhibits a darkening of the skin (falling L values) and generates a more natural hue of the skin, confirmed by the increasing a values of a red-brown shift. Glucuronolactone is a self-tanning substance.

In mixture P1, a red-brown shift is likewise observed (Δa=0.25 after one day, Δa=1.48 after 5 days, Δa=2.28 after 11 days). Glucuronolactone in the mixture therefore causes a colour shift of the tanning result towards a more natural hue, i.e. towards a hue which corresponds to natural tanning.

Δa after one day is calculated as follows:

Δa=(Δa P1 1 D1+Δa P1 2 D1+Δa P1 3 D1)/3)−(Δa R1D1+Δa R2D1+Δa R3D1)/3).

### EXAMPLE 4

### In-Vitro Investigation of DHA and DHA/Glucuronolactone

90 mg of DHA (1 mmol) and 176 mg of glucuronolactone (1 mmol) are dissolved in 94 ml of ethylene glycol and 6 ml of water (potassium hydrogenphthalate—buffered to pH 7) with 252 mg of DL-lysine (2 mmol) and stirred at 37° C. (=Liquidskin model). The L-a-b values of the solution are subsequently measured by means of UV-VIS spectrometer (Varian Cary-100) after 1 hour, 6 hours and 24 hours and compared with DHA/lysine (90 mg/146 mg) in the Liquidskin model.

### Results:

After one hour:

| | L | a | b |
|---|---|---|---|
| DHA | 99.4 | 0.4 | 1.3 |
| DHA/glucuronolactone | 96.3 | 2.4 | 37.3 |

After 6 hours:

| | L | a | b |
|---|---|---|---|
| DHA | 86.1 | 13.4 | 39.9 |
| DHA/glucuronolactone | 78.4 | 26.4 | 100.6 |

After 24 hours:

| | L | a | b |
|---|---|---|---|
| DHA | 61.3 | 31.7 | 75.0 |
| DHA/glucuronolactone | 44.1 | 39.0 | 73.3 |

The results of the in-vitro test confirm that the DHA/glucuronolactone mixture generate a darker value (lower L value). The higher a value confirms the red/brown shift and an accompanying colour impression which corresponds to more natural tanning.

### FORMULATION EXAMPLE 1

### O/W Tanning Cream

| | | | |
|---|---|---|---|
| Constituents/ | Source of | | [% by |
| trade name | supply | INCI | wt.] |

A

| | | | |
|---|---|---|---|
| Marlipal 1618/11 | (1) | CETEARETH-11 | 3 |
| Lanette O | (2) | CETEARYLALCOHOL | 7 |
| Luvitol EHO | (3) | CETEARYLOCTANOATE | 5 |
| Tegosoft TN | (4) | C12-15 | 2.5 |
| | | ALKYLBENZOATE | |
| Miglyol 812 N | (1) | CAPRYLIC/CAPRIC | 2.5 |
| | | TRIGLYCERIDE | |
| Propyl | (5) | PROPYLPARABEN | 0.05 |
| 4-hydroxybenzoate | | | |

B

| | | | |
|---|---|---|---|
| 1,2-Propanediol | (5) | PROPYLENE GLYCOL | 4 |
| Methyl | (5) | METHYLPARABEN | 0.15 |
| 4-hydroxybenzoate | | | |
| Water, | | AQUA (WATER) | to 100 |
| demineralised | | | |
| Glucuronolactone | (6) | Glucuronolactone | 5 |

C

| | | | |
|---|---|---|---|
| Dihydroxyacetone | (7) | DIHYDROXYACETONE | 5 |
| Water, | | | 10 |
| demineralised | | | |
| Total | | | 100.00 |

### Preparation Process:

Firstly, phase A is warmed to 75° C. and phase B to 80° C. Phase B is then slowly added to phase A with stirring and stirred until a homogeneous mixture forms. After homogenisation, phase C is added to the formulation at 40° C.

### Sources of Supply:

(1) Sasol Germany GmbH (2) Cognis GmbH (3) BASF AG (4) Degussa-Goldschmidt AG (5) Merck KGaA/Rona® (6) Pfaltz&Bauer, Inc., (7) Merck KGaA/Rona®

### FORMULATION EXAMPLE 2

### O/W Tanning Cream

| | | | |
|---|---|---|---|
| Constituents/ | Source of | | [% by |
| trade name | supply | INCI | wt.] |

A

| | | | |
|---|---|---|---|
| Tego Care 150 | (1) | GLYCERYL STEARATE, | 8 |
| | | STEARETH-25-CETETH-20, | |
| | | STEARYL ALCOHOL | |
| Miglyol 812 N | (2) | CAPRYLIC/CAPRIC | 3 |
| | | TRIGLYCERIDE | |
| Isopropyl myristate | (3) | ISOPROPYL MYRISTATE | 2 |
| Paraffin liquid | (4) | PARAFFINUM LIQUIDUM | 12 |
| | | (MINERAL OIL) | |
| Paraffin | (4) | PARAFFIN | 2 |
| Propyl | (4) | PROPYLPARABEN | 0.15 |
| 4-hydroxybenzoate | | | |

B

| | | | |
|---|---|---|---|
| 1,2-Propanediol | (4) | PROPYLENE GLYCOL | 4 |
| Sorbitol F liquid | (4) | SORBITOL | 2 |
| Methyl | (4) | METHYLPARABEN | 0.05 |
| 4-hydroxybenzoate | | | |
| Glucuronolactone | (5) | Glucuronolactone | 5 |
| Water, | | AQUA (WATER) | to 100 |
| demineralised | | | |

C

| | | | |
|---|---|---|---|
| Dihydroxyacetone | (6) | DIHYDROXYACETONE | 5 |
| Water, | | | 11.8 |
| demineralised | | | |

D

| | | |
|---|---|---|
| Perfume (q.s.) | PARFUM | 0.50 |
| Total | | 100.00 |

### Preparation Process:

Firstly, phases A and B are warmed separately to 75° C. Phase A is then slowly added to phase B with careful stirring. The mixture is homogenised at 65° C. for one minute. The mixture is subsequently cooled to 40° C. with stirring, and phase C is added with stirring, the mixture is cooled to 35° C., and phase D is added, and cooling is continued.

### Sources of Supply:

(1) Degussa-Goldschmidt AG, (2) Sasol Germany GmbH, (3) Cognis GmbH, (4) Merck KGaA/Rona®, (5) Pfaltz&Bauer, Inc., (6) Merck KGaA/Rona®

### FORMULATION EXAMPLE 3

### O/W Tanning Cream

| | | | |
|---|---|---|---|
| Constituents/ | Source of | | [% by |
| trade name | supply | INCI | wt.] |

A

| | | | |
|---|---|---|---|
| Tego Care 150 | (1) | GLYCERYL STEARATE, | 8 |
| | | STEARETH-25, | |
| | | CETETH-20, | |
| | | STEARYL ALCOHOL | |
| Lanette O | (2) | CETEARYL ALCOHOL | 1.5 |
| Luvitol EHO | (3) | CETEARYL OCTANOATE | 5 |
| Miglyol 812 N | (4) | CAPRYLIC/CAPRIC | 5 |
| | | TRIGLYCERIDE | |
| Paraffin liquid | (5) | PARAFFINUM LIQUIDUM | 3 |
| | | (MINERAL OIL) | |
| AbilWax 2434 | (1) | STEAROXY | 1.6 |
| | | DIMETHICONE | |
| Dow Corning | (6) | DIMETHICONE | 0.5 |
| 200 Fluid (350 cs) | | | |
| Propyl | (5) | PROPYLLPARABEN | 0.05 |
| 4-hydroxybenzoate | | | |

B

| | | | |
|---|---|---|---|
| 1,2-Propanediol | (5) | PROPYLENE GLYCOL | 3 |
| Methyl | (5) | METHYLPARABEN | 0.15 |
| 4-hydroxybenzoate | | | |
| Water, | | AQUA (WATER) | to 100 |
| demineralised | | | |

C

| | | | |
|---|---|---|---|
| Dihydroxyacetone | (8) | DIHYDROXYACETONE | 5 |
| Probiol L 05018 | (7) | AQUA, ALCOHOL DENAT, | 5 |
| (Empty liposomes) | | LECITHIN, GLYCERINE, | |
| | | DISODIUM PHOSPHATE | |
| Water, | | AQUA (WATER) | 10.00 |
| demineralised | | | |
| Glucuronolactone | (9) | Glucuronolactone | 5 |
| Total | | | 100.00 |

### Preparation Process:

Firstly, phases A and B are warmed to 80° C. Phase B is then slowly added to phase A with stirring and homogenised. The mixture is then cooled, and phase C is added at 40° C.

### Sources of Supply:

(1) Degussa-Goldschmidt AG, (2) Cognis GmbH, (3) BASF AG, (4) Sasol Germany GmbH, (5) Merck KGaA/Rona®, (6) Dow Corning, (7) Kuhs GmbH & Co. KG, (8) Merck KGaA/Rona®, (9) Pfaltz&Bauer, Inc.

### FORMULATION EXAMPLE 4

### O/W Tanning Lotion

| | | | |
|---|---|---|---|
| Constituents/ | Source of | | [% by |
| trade name | supply | INCI | wt.] |

A

| | | | |
|---|---|---|---|
| Montanov 68 | (1) | CETEARYL ALCOHOL, | 4 |
| | | CETEARYL GLUCOSIDE | |
| Span 60 | (2) | SORBITAN STEARATE | 1.5 |
| Lanette O | (3) | CETEARYL ALCOHOL | 1 |
| Cosmacol ELI | (4) | C12-13 ALIKYL LACTATE | 2 |
| Arimol HD | (2) | ISOHEXADECANE | 1 |
| Paraffin | (5) | PARAFFINUM LIQUIDUM | 3 |
| highly liquid | | (MINERAL OIL) | |
| Dow Corning | (6) | CYCLOMETHICONE, | 2 |
| 9050 Silicone | | | |
| Elastomer Blend | | DIMETHICONE | |
| | | CROSSPOLYMER | |
| RonaCare ® | (5) | TOCOPHERYL ACETATE | 0.5 |
| Tocopherol | | | |
| Acetate | | | |
| Propyl | (5) | PROPYLPARABEN | 0.05 |
| 4-hydroxybenzoate | | | |

B

| | | | |
|---|---|---|---|
| RonaCare ® Ectoin | (5) | ECTOIN | 0.5 |
| Glycerol, anhydrous | (5) | GLYCERINE | 2 |
| Water, | | AQUA (WATER) | to 100 |
| demineralised | | | |
| Methyl | (5) | METHYLPARABEN | 0.15 |
| 4-hydroxybenzoate | | | |
| Glucuronolactone | (11) | Glucuronolactone | 5 |

C

| | | | |
|---|---|---|---|
| Rhodicare S | (7) | XANTHAN GUM | 0.2 |

D

| | | | |
|---|---|---|---|
| Probiol L 05018 | (8) | AQUA, ALCOHOL DENAT, | 5 |
| (Empty liposomes) | | LECITHIN, GLYCERINE, | |
| | | DISODIUM PHOSPHATE | |
| Dihydroxyacetone | (10) | DIHYDROXYACETONE | 5 |
| Water, | | AQUA (WATER) | 10 |
| demineralised | | | |

E

| | | | |
|---|---|---|---|
| Fragrance | (9) | PARFUM | 0.2 |
| Cucumber | | | |
| Total | | | 100.00 |

### Preparation Process:

Firstly, phases A and B are mixed separately and warmed to 75° C. Phase C is then added to phase B and added to phase A with stirring. The mixture is homogenised. The mixture is then cooled with stirring, and phases D and E are added at 40° C.

### Sources of Supply:

(1) Seppic (2) Uniqema (3) Cognis GmbH (4) Condea Chinica D.A.C.S.p.A. (5) Merck KGaA/Rona® (6) Dow Corning (7) Rhodia GmbH (8) Kuhs GmbH & Co. KG (9) Drom (10) Merck KGaA/Rona® (11) Pfaltz&Bauer, Inc.

### FORMULATION EXAMPLE 5

### Mild Transparent W/O Tanning Lotion

| | | | |
|---|---|---|---|
| Constituents/ | Source of | | |
| trade name | supply | INCI | [% by wt.] |

A

| | | | |
|---|---|---|---|
| Dow Corning | (1) | CYCLOMETHICONE, | 23.6 |
| 3225 C | | DIMETHICONE | |
| | | COPOLYOL | |
| Propyl | (2) | PROPYLPARABEN | 0.05 |
| 4-hydroxybenzoate | | | |

B

| | | | |
|---|---|---|---|
| Dihydroxyacetone | (3) | DIHYDROXYACETONE | 3 |
| Glucuronolactone | (4) | Glucuronolactone | 2 |
| Methyl | (2) | METHYLPARABEN | 0.15 |
| 4-hydroxybenzoate | | | |
| 1,2-Propanediol | (2) | PROPYLENE GLYCOL | 35.9 |
| Water, | | AQUA (WATER) | to 100 |
| demineralised | | | |
| Total | | | 100.00 |

### Preparation Process:

Firstly, phase B is dissolved and then added to phase A. The pH is adjusted to the value pH=6.0 using sodium hydroxide solution or citric acid.

### Sources of Supply:

(1) Dow Corning (2) Merck KGaA/Rona® (3) Merck KGaA/Rona® (4) Pfaltz&Bauer, Inc.,

### FORMULATION EXAMPLE 6

### O/W Tanning Cream with UV A/B Protection

| | | | |
|---|---|---|---|
| Constituents/ | Source of | | [% by |
| trade name | supply | INCI | wt.] |

A

| | | | |
|---|---|---|---|
| Eusolex ® 2292 | (1) | ETHYLHEXYL | 3 |
| | | METHOXYCINNAMATE, | |
| | | BHT | |
| Eusolex ® 4360 | (1) | BENZOPHENONE-3 | 0.5 |
| Tego Care 150 | (2) | GLYCERYL STEARATE, | 8 |
| | | STEARETH-25, | |
| | | CETETH-20, | |
| | | STEARYL ALCOHOL | |
| Lanette O | (3) | CETEARYL ALCOHOL | 1.5 |
| Luvitol EHO | (4) | CETEARYL OCTANOATE | 5 |
| Miglyoll 812 N | (5) | CAPRYLIC/CAPRIC | 5 |
| | | TRIGLYCERIDE | |
| Paraffin liquid | (1) | PARAFFINUM LIQUIDUM | 3 |
| | | (MINERAL OIL) | |
| Abil-Wax 2434 | (2) | STEAROXY | 1.6 |
| | | DIMETHICONE | |
| Dow Corning 200 | (6) | DIMETHICONE | 0.5 |
| Fluid (350 cs) | | | |
| Propyl | (1) | PROPYLPARABEN | 0.05 |
| 4-hydroxybenzoate | | | |

B

| | | | |
|---|---|---|---|
| 1,2-Propanediol | (1) | PROPYLENE GLYCOL | 3 |
| Methyl | (1) | SODIUM | 0.17 |
| 4-hydroxybenzoate | | METHYLPARABEN | |
| sodium salt | | | |
| Water, | | AQUA (WATER) | to 100 |
| demineralised | | | |
| Glucuronolactone | (7) | Glucuronolactone | 5 |

C

| | | | |
|---|---|---|---|
| Dihydroxyacetone | (8) | DIHYDROXYACETONE | 5 |
| Water, | | | 10 |
| demineralised | | | |
| Total | | | 100.00 |

### Preparation Process:

Firstly, phases A and B are mixed separately and warmed to 80° C. Phase B is then slowly added to phase A with stirring. The mixture is homogenised and cooled to 40° C., and phase C is added, then cooled to room temperature.

### Sources of Supply:

(1) Merck KGaA/Rona®, (2) Degussa-Goldschmidt AG, (3) Cognis GmbH, (4) BASF AG, (5) Sasol Germany GmbH, (6) Dow Corning, (7) Pfaltz&Bauer, Inc., (8) Merck KGaA/Rona®

### FORMULATION EXAMPLE 8

### O/W Shimmering Tanning Lotion

| | | | |
|---|---|---|---|
| Constituents/ | Source of | | [% by |
| trade name | supply | INCI | wt.] |

A

| | | | |
|---|---|---|---|
| Montanov 68 | (1) | CETEARYL ALCOHOL, | 4 |
| | | CETEARYL GLUCOSIDE | |
| Span 60 | (2) | SORBITAN STEARATE | 1.5 |
| Lanette O | (3) | CETEARYL ALCOHOL | 1 |
| Cosmacol ELI | (4) | C12-13 ALKYL LACTATE | 3 |
| Cosmacol EMI | (4) | DI-C12-13 ALKYL | 1 |
| | | MALATE | |
| Dow Corning | (5) | CYCLOMETHICONE, | 1 |
| 9040 Silicone | | | |
| Elastomer Blend | | DIMETHICONE | |
| | | CROSSPOLYMER | |
| Arlamol HD | (2) | ISOHEXADECANE | 3 |
| RonaCare ® | (6) | TOCOPHERYL ACETATE | 0.5 |
| Tocopherol Acetate | | | |
| Propyl | (6) | PROPYLPARABEN | 0.05 |
| 4-hydroxybenzoate | | | |

B

| | | | |
|---|---|---|---|
| RonaCare ® Ectoin | (6) | ECTOIN | 0.5 |
| Colorona ® | (6) | MICA, CI 77891 | 2 |
| Red Gold | | (TITANIUM DIOXIDE), | |
| | | CI 77491(ION OXIDES) | |
| Glycerol, anhydrous | (6) | GLYCERIN | 2 |
| Caramel 250 | (7) | CARAMEL | 1 |
| FD&C Yellow | (8) | CI 15985 | 0.01 |
| No6 W082 | | | |
| Water, | | AQUA (WATER) | to 100 |
| demineralised | | | |
| Methyl | (6) | METHYLPARABEN | 0.15 |
| 4-hydroxybenzoate | | | |
| Glucuronolactone | (11) | Glucuronolactone | 5 |

C

| | | | |
|---|---|---|---|
| Sepigel 305 | (1) | LAURETH-7, POLYACRYL- | 0.5 |
| | | AMIDE, C13-14 | |
| | | ISOPARAFFIN | |

D

| | | | |
|---|---|---|---|
| Dihydroxyacetone | (10) | DIHYDROXYACETONE | 5 |
| Water, | | | 10 |
| demineralised | | | |

E

| | | | |
|---|---|---|---|
| Fragrance Babylon | (9) | PARFUM | 0.2 |
| Total | | | 100.00 |

### Preparation Process:

Firstly, phases A and B are warmed separately to 75° C. Phase A is then slowly added to phase B with stirring. Phase C is added to A/B at 60° C., and the mixture is homogenised. The mixture is subsequently cooled to 40° C., and phases D and E are added successively.

### Sources of Supply:

(1) Seppic (2) Uniqema (3) Cognis GmbH (4) Condea Chimica D.A.C.S.p.A. (5) Dow Corning (6) Merck KGaA/Rona® (7) D. D. Williamson (8) Les Colorants Wackherr SA (9) Drom (10) Merck KGaA/Rona® (11) Pfaltz&Bauer, Inc.

1. A method for enhancing the tanning achieved by dihydroxyacetone or a mixture of self-tanning substances comprising dihydroxyacetone, said method comprising using at least one compound of the formula I 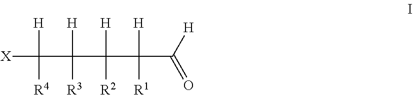
where R¹, R², R³ and R⁴ each stand, independently of one another, for H, OH, O⁻Kt⁺, OAlk, NH₂, NHCOCH₃ or OAcy,
where X stands for COOH or CHO,
where Kt⁺ stands for Na⁺, K⁺, NH₄⁺,
where the C atoms to which R¹, R², R³ or R⁴ are bonded may each, independently of one another, be in the R or S configuration,
where the compounds of the formula I can be in an open-chain form or in a furanoid or pyranoid lactone and/or lactol form,
and/or salt thereof as a tanning enhancer.

2. A method of tanning comprising applying to the skin at least one compound of the formula I 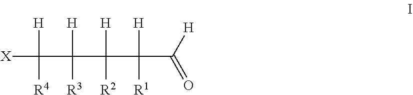
where R¹, R², R³ and R⁴ each stand, independently of one another, for H, OH, O⁻Kt⁺, OAlk, NH₂, NHCOCH₃ or OAcy,
where X stands for COOH or CHO,
where Kt⁺ stands for Na⁺, K⁺, NH₄⁺,
where the C atoms to which R¹, R², R³ or R⁴ are bonded may each, independently of one another, be in the R or S configuration,
where the compounds of the formula I can be in an open-chain form or in a furanoid or pyranoid lactone and/or lactol form,
and/or salt thereof
as a self-tanning substance.

3. A method of reducing contrast in tanning achieved by a mixture or preparation comprising at least one self-tanning substance, said method comprising using at least one compound of the formula I 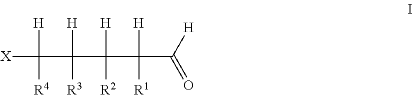
where R¹, R², R³ and R⁴ each stand, independently of one another, for H, OH, O⁻Kt⁺, OAlk, NH₂, NHCOCH₃ or OAcy,
where X stands for COOH or CHO,
where Kt⁺ stands for Na⁺, K⁺, NH₄⁺,
where the C atoms to which R¹, R², R³ or R⁴ are bonded may each, independently of one another, be in the R or S configuration,
where the compounds of the formula I can be in an open-chain form or in a furanoid or pyranoid lactone and/or lactol form,
and/or salt thereof in said mixture or preparation comprising at least one self-tanning substance, as contrast reduction agent.

4. A method for modulating the hue of a tan achieved by use of dihydroxyacetone comprising using at least one compound of the formula I 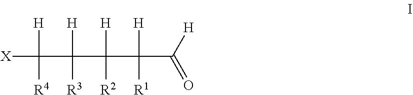
where R¹, R², R³ and R⁴ each stand, independently of one another, for H, OH, O⁻Kt⁺, OAlk, NH₂, NHCOCH₃ or OAcy,
where X stands for COOH or CHO,
where Kt⁺ stands for Na⁺, K⁺, NH₄⁺,
where the C atoms to which R¹, R², R³ or R⁴ are bonded may each, independently of one another, be in the R or S configuration,
where the compounds of the formula I can be in an open-chain form or in a furanoid or pyranoid lactone and/or lactol form,
and/or salt thereof for modulation of the hue achieved in the case of tanning with dihydroxyacetone or by the mixture or preparation comprising dihydroxyacetone.

5. A method according to claim 1,
characterised in that
R¹, R², R³ and R⁴ each stand, independently of one another, for H, OH, O⁻Kt⁺, OAlk, or OAcy.

6. A method according to claim 1,
characterised in that
R¹, R², R³ and R⁴ stand for OH.

7. A method according to claim 1,
characterised in that
the compound of the formula I is in a lactol form.

8. A method according to claim 1,
characterised in that
X stands for COOH.

9. A method according to claim 1,
characterised in that
the compound of the formula I is in a lactone form.

10. A method according to claim 1,
characterised in that
the dihydroxyacetone-containing mixture of self-tanning substances comprises at least one further self-tanning substance selected from the group glycerolaldehyde, hydroxymethylglyoxal, γ-dialdehyde, erythrulose, 6-aldo-D-fructose, ninhydrin, 5-hydroxy-1,4-naphtoquinone (juglone) and/or 2-hydroxy-1,4-naphtoquinone (lawsone).

11. A composition comprising at least one vehicle which is suitable for cosmetic, pharmaceutical, dermatological preparations, at least dihydroxyacetone and at least one compound of the formula I according to claim 1.

12. A composition according to claim 11,
characterised in that
the preparation comprises at least one compound of the formula I in an amount of 0.01 to 20% by weight, based on the total amount of the preparation.

13. A composition according to claim 11,
characterised in that
the preparation comprises at least dihydroxyacetone in an amount of 0.01 to 20% by weight, based on the total amount of the preparation.

14. A composition according to claim 11,
characterised in that
the percent by weight ratio of least one compound of the formula I to dihydroxyacetone is 1:20 to 20:1.

15. Process for the preparation of a preparation, in particular according to claim 11,
in which at least one compound of the formula I is mixed together with at least one vehicle which is suitable for cosmetic, pharmaceutical, dermatological preparations and optionally assistants and/or fillers or self-tanning substances, and dihydroxyacetone is added.
